(19) United States
(12) Patent Application Publication
(10) Pub. No.: US 2010/0035337 A1
(43) Pub. Date: Feb. 11, 2010

(54) Bioreactor with window

(75) Inventors: Detlef Bahnemann, Garbsen (DE); Reinhard Baumfalk, Goettingen (DE); Daniel Riechers, Hanover (DE); Thomas Scheper, Hanover (DE)

Correspondence Address: CASELLA & HESPOS, 274 MADISON AVENUE, NEW YORK, NY, 10016, US

(73) Assignee: Sartorius Stedim Biotech GmbH, Goettingen (DE)

(21) Appl. No.: 12/460,314

(22) Filed: Jul. 16, 2009

(30) Foreign Application Priority Data
Aug. 8, 2008 (DE) 10 2008 036 934.9

(51) Int. Cl. C12M 3/00

(52) U.S. Cl. 435/292.1

(57) ABSTRACT

Bioreactor having at least one transparent window (5, 12, 13), the inner side (6) of which can be touched by a medium that can be arranged in a reactor interior (3), wherein the window (5, 12, 13) has a photocatalytic coating (7) on its inner side (6) facing the reactor interior (3), which coating can be activated by at least one light source from the outer side remote from the inner side (6).

### BACKGROUND OF THE INVENTION

1. Field of the Invention

The invention relates to a bioreactor having at least one transparent window, the inner side of which can be touched by a medium that can be arranged in a reactor interior.

2. Description of the Related Art

Noninvasive optical measurement methods are increasingly gaining in importance for the online monitoring of bioprocesses. The particular advantage of these measurement methods is based on the fact that suitable sensor systems can be coupled via optical windows to bioreactors or fermenters, for example, whereby any risk of contamination arising as a result of offline sampling is precluded. A further advantage is that optical measurement methods generally operate very rapidly and continuously and, on account of the weak interactions between electromagnetic radiation and matter, nondestructively.

Particular requirements are made of the optical windows required for these measurement methods. Firstly, they have to be transparent to the wavelength range used and, secondly, they must not be blocked by radiation-absorbing contamination such as e.g. eukaryotic or prokaryotic cells or culture medium constituents during measurement operation.

EP 1 269 243 B1 discloses a microscope probe which can be inserted into a connection port of a fermenter (bioreactor) and which enables monitoring or control of the bioprocess directly in the culture broth or the medium situated in the reactor interior.

The known apparatus, which has proved to be worthwhile in principle, has the disadvantage that the sample zone of the microscope probe that comes into contact with the medium to be examined has to be cleaned relatively laboriously by means of cleaning liquid supplied externally.

DE 10 2004 019 234 B3 discloses a bioreactor for cultivating phototropic microorganisms, which has an illumination device having a plurality of light sources which are embodied as fluorescent tubes and which expose light-transmissive gas-introduction tubes and a light-transmissive standpipe for carrying out the phototropic reactions of the microorganisms.

The known apparatus, which has proved to be worthwhile in principle, has the disadvantage that the light transmissivity of the gas-introduction tubes and standpipe that come into contact with the culture medium is reduced by deposits on the inner sides facing the reactor interiors over the course of the operating period and necessitate costly cleaning procedures.

WO 97/07 069 A1 discloses a self-cleaning glass that can be used for example for windows of buildings or for windshields of vehicles. The glass has an optically transparent coating containing a photocatalyst, the photocatalytic effect of which is initiated by action of solar or UV radiation and leads to a self-cleaning of the glass by a photochemical oxidation process. The known glass has titanium dioxide particles in its coating in order to achieve its photocatalytic effect. The person skilled in the art cannot infer from this document any indication about a use of a coating of this type in a bioreactor.

Furthermore, methods for producing titanium dioxide coatings are known for example from DE 19 25 606 B2 and DE 102006 044 076 A1.

It is an object of the present invention, therefore, to lengthen the service life of optical windows in bioreactors and to manage as far as possible without interrupting the operating process for a time-consuming cleaning of the window side that is touched by media.

### SUMMARY OF THE INVENTION

The object is achieved in conjunction with a bioreactor having at least one transparent window in that the window has a photocatalytic coating on its inner side facing the reactor interior, which coating can be activated by at least one light source from the outer side remote from the inner side.

By virtue of the fact that the photocatalytic coating of the window that is touched by media can be activated by a light source, the adhering of media particles is prevented or particles or substances already adhering to the coating of the window are removed again by a light-initiated photocatalytic oxidation. Thus, a significantly improved service life of the window and thus a significantly improved long-term performance of the optical sensor systems are achieved.

The light source is preferably a UV light source.

In one preferred embodiment, the invention can be applied to bioreactors for cultivating phototropic microorganisms, so-called photobioreactors. In this case, at least one portion of the walls of the reactor space is equipped with a transparent window, wherein the window has a photocatalytic coating on its inner side facing the reactor interior, which coating can be activated by at least one light source from the outer side remote from the inner side. Here a window according to the invention should quite generally also be understood to mean a transparent region in the wall of the reactor interior.

It has surprisingly been found that during the operation of photobioreactors equipped in this way over a period of 20 days, the radiation intensity remains unchanged and no visible overgrowth on the window could be detected.

In accordance with one preferred embodiment of the invention, the coating is embodied as a nanocrystalline titanium dioxide coating. In this case, the coating is arranged on a window composed of a low-alkali glass. Suitable glasses are for example quartz, sapphire, low-sodium float and borosilicate glass.

The window as optical element can be present for example in the form of a prism or crystal having attenuated total reflection (ATR crystal).

Owing to their chemical resistance and their good thermal stability, the abovementioned glasses are particularly suitable for a coating in the dipping bath (dip coating) with subsequent calcination at a maximum temperature of 500° C.

According to a further preferred embodiment of the invention, the UV light can be generated by at least one UV-LED having an emission maximum of between 200 and 400 nm, for example 360 nm.

The UV light required for the initiation of the photocatalytic oxidation can be supplied by a UV-LED, adjacent to the window, in a space-saving manner and relatively simply without major losses for the active coating. The wavelength used is particularly suitable for the chosen coating with TiO₂ nanoparticles.

In accordance with a further preferred embodiment of the invention, a probe receptacle projecting into the reactor interior through the wall thereof is disposed upstream of the window. A measuring probe and/or the at least one UV-LED can be inserted into the probe receptacle.

A measuring probe can be inserted into the probe receptacle without any risk of contamination, which measuring probe can be exchanged for an insertable UV-LED as necessary for self-cleaning of the window. It is particularly expedient, however, to arrange the UV-LED directly in the measuring probe, whereby mutual exchange can be dispensed with. Moreover, the UV illumination intervals can thereby be increased without any problems.

According to a further preferred embodiment of the invention, two windows arranged at a distance and parallel to one another delimit a cuvette gap of a measuring probe embodied as a transmission probe and projecting into the reactor interior, wherein each of the windows is assigned a UV-LED. In this case, an optical signal can be fed via a deflection prism to the first window and further via the cuvette gap, filled by the surrounding medium, via the second window to an optical evaluation unit. Particularly advantageously, the first window can be formed by a coated partial region of the deflection prism. This is especially space-saving.

The optical signal is conducted via a first optical fiber to the deflection prism and via a second optical fiber, disposed ahead of the second window, to the optical evaluation unit, which is embodied as a spectrometer, for example.

Further embodiments are for example windows for 90° and 180° scattered light sensors for turbidity measurement, by means of which biomass determinations are performed.

In accordance with a further preferred embodiment of the invention, at least the reactor container having the reactor interior is embodied as a disposable container.

Particularly when using bioreactors as disposable bioreactors, such as are often used with a flexible wall as bags, the use of a window coated according to the invention is of great importance since the service life of the entire bioreactor is considerably increased.

Further features of the invention emerge from the following detailed description and the accompanying drawings illustrating preferred embodiments of the invention by way of example.

### BRIEF DESCRIPTION OF THE DRAWINGS

FIG. 1 shows a side view of a bioreactor with a probe receptacle, projecting into the reactor interior, with an optical window and inserted UV-LED;

FIG. 2 shows a side view of a bioreactor with a probe receptacle, projecting into the reactor interior, with an optical window and inserted measuring probe;

FIG. 3 shows an enlarged illustration of a measuring probe embodied as a transmission probe; and

FIG. 4 shows a side view of a bioreactor as a detail with the transmission probe from FIG. 3 projecting into the reactor interior.

### DESCRIPTION OF THE ILLUSTRATIVE EMBODIMENTS

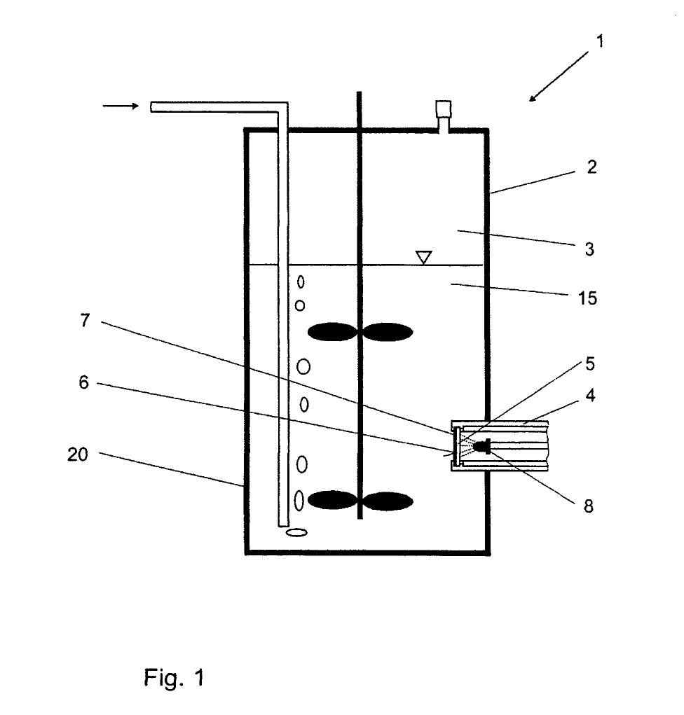

Turning to FIG. 1, a bioreactor 1 essentially comprises a reactor interior 3 enclosed by a flexible wall 2, and a probe receptacle 4 with an optical window 5.

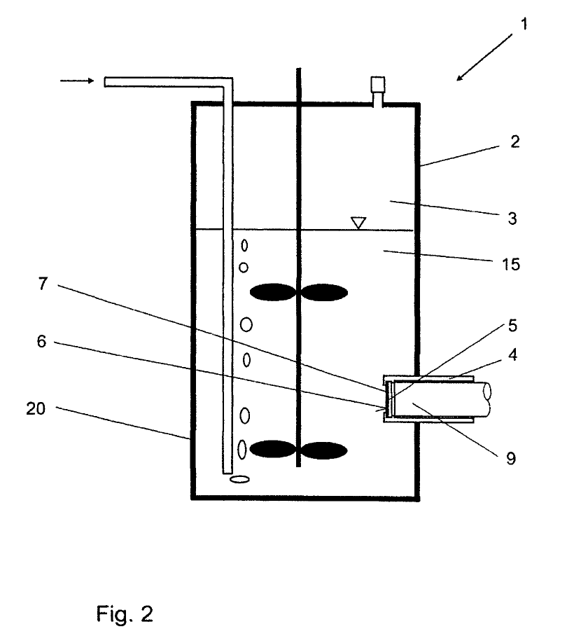

The probe receptacle 4 of the embodiment according to FIGS. 1 and 2 is closed off toward the reactor interior 3 by the optical window 5, which has a photocatalytic coating 7 on its inner side 6 facing the reactor interior 3. The coating is embodied as a nanocrystalline titanium dioxide coating (TiO₂). The optical window 5 is composed of a borosilicate glass.

A UV-LED 8 can be inserted into the probe receptacle 4 in order to activate the photocatalytic coating 7, and a measuring probe 9 can be inserted in order to carry out the measuring operation. However, the UV-LED 8 can also be integrated into the measuring probe 9.

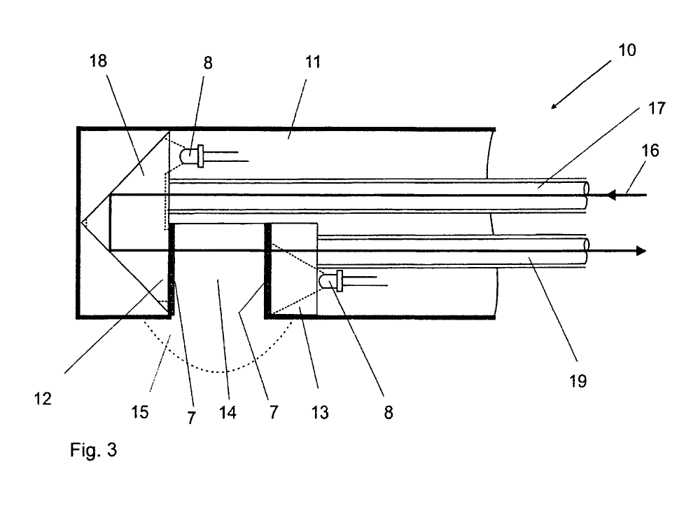

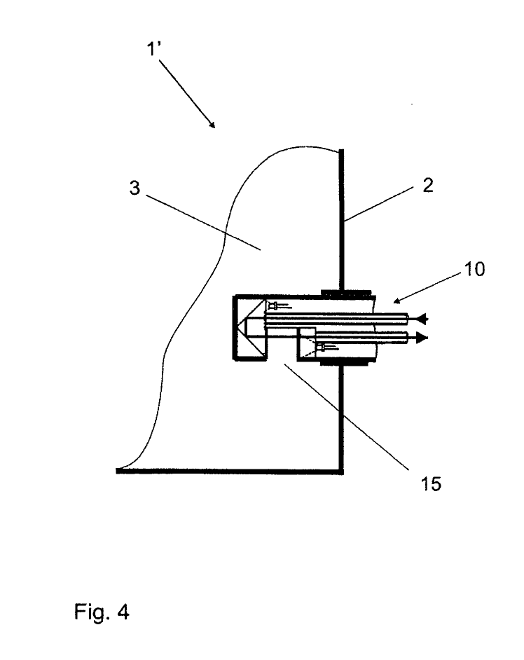

In accordance with the exemplary embodiment in FIGS. 3 and 4, the measuring probe 9 is embodied as a transmission probe 10, which projects by its free end 11 into the reactor interior 3 of the bioreactor 1′ and has a first optical window 12 and, at a distance, a second optical window 13, which delimit a cuvette gap 14 filled by the surrounding medium 15 of the reactor interior 3.

In this embodiment, an optical signal 16 can be fed via a first optical fiber 17 to a deflection prism 18, adjacent to the first window 12, in a manner bridging the cuvette gap 14 to a second optical fiber 19, disposed ahead of the second window 13, and further to an optical evaluation unit (not illustrated), embodied as a spectrometer, for example. The first window 12 in the exemplary embodiment according to FIGS. 3 and 4 is formed by a coated partial region of the deflection prism 18.

The bioreactor 1, 1′ or its reactor container 20 is embodied as a disposable container having the flexible wall 2.

The following procedure can be adopted for producing the coated window 5, 12, 13:

1. Catalyst Synthesis:

Si binder is initially introduced and stirred with addition of 65% strength HNO₃. The pH of the binder is set in the acid range with slow addition of Ti precursor in the ice bath with subsequent stirring, while an organic solvent is added in a manner mixed slowly by means of a dropping funnel in the ice bath. After stirring overnight, the viscosity is measured for monitoring purposes.

2. Coating Process:

The aim of the coating process is to achieve a transparent, photocatalytically active, stable film layer. The coating is effected by dip coating. A low-alkali glass, for example borosilicate glass, is used as material of the window 5, 12, 13 or substrate. The substrate is pretreated by boiling in concentrated sulfuric acid and subsequent boiling in sodium hydroxide solution.

In this case, the following coating parameters are used in the exemplary embodiment:
dipping rate: 10-30 mm/s
holding time: 10 to 20 s
pulling rate: 1 to 3 mm/s
relative air humidity: 20 to 40%

3. Calcination

The following parameters were used for calcination:
maximum temperature: 500° C.
heating up: within 30 to 60 min
maintaining the maximum temperature: 60 min
cooling down: over a number of hours

What is claimed is:

1. Bioreactor having at least one transparent window (5, 12, 13), the inner side (6) of which can be touched by a medium that can be arranged in a reactor interior (3), characterized in that the window (5, 12, 13) has a photocatalytic coating (7) on its inner side (6) facing the reactor interior (3), which coating can be activated by at least one light source from the outer side remote from the inner side (6).

2. Bioreactor according to claim 1, characterized in that the window (5, 12, 13) is part of the reactor interior (3) of a photobioreactor.

3. Bioreactor according to claim 1, characterized in that the coating (7) is embodied as a nanocrystalline titanium dioxide coating.

4. Bioreactor according to claim 1, characterized in that the window (5, 12, 13) is formed from a low-alkali glass.

5. Bioreactor according to claim 1, characterized in that the light source emits UV light that can be generated by at least one UV-LED (8).

6. Bioreactor according to claim 5, characterized in that the UV-LED (8) has an emission maximum of between 200 and 400 nm.

7. Bioreactor according to claim 1, characterized in that a probe receptacle (4) projecting into the reactor interior (3) through the wall (2) thereof is disposed upstream of the window (5).

8. Bioreactor according to claim 7, characterized in that a measuring probe (9) and/or the at least one UV-LED (8) can be inserted into the probe receptacle (4).

9. Bioreactor according to claim 1, characterized in that two windows (12, 13) arranged at a distance and parallel to one another delimit a cuvette gap (14) of a transmission probe (10) projecting into the reactor interior (3).

10. Bioreactor according to claim 9, characterized in that each of the windows (12, 13) is assigned a UV-LED (8).

11. Bioreactor according to claim 10, characterized in that an optical signal (16) can be fed via a deflection prism to the first window (12) and further via the cuvette gap (14), filled by the surrounding medium (15), via the second window (13) to an optical evaluation unit.

12. Bioreactor according to claim 11, characterized in that the optical signal (16) can be conducted via a first optical fiber (17) to the deflection prism (18) and via a second optical fiber (19), disposed ahead of the second window (13), to the optical evaluation unit.

13. Bioreactor according to claim 1, characterized in that a reactor container (20) having the reactor interior (3) is embodied as a disposable container.
